(19) United States
(12) Patent Application Publication
(10) Pub. No.: US 2003/0135080 A1
(43) Pub. Date: Jul. 17, 2003

(54) Metathesis process for converting short chain olefins to longer chain olefins

(76) Inventors: Jan Mattheus Botha, Sasolburg (ZA); Jan Petrus Karel Reynhardt, Sasolburg (ZA); Charl Van Schalkwyk, Sasolburg (ZA); Hermanus Cornelius Moolman Vosloo, Potchefstroom (ZA)

Correspondence Address: Fish & Richardson, 225 Franklin Street, Boston, MA, 02110-2804, US

(21) Appl. No.: 10/168,255

(22) PCT Filed: Dec. 21, 2000
(86) PCT No.: PCT/ZA00/00258

(51) Int. Cl. C07C 6/04; C07C 6/00

(52) U.S. Cl. 585/646; 585/647

(57) ABSTRACT

The invention provides a homogeneous metathesis process for converting C₄ to C₁₀ olefins in a Fischer-Tropsch derived feedstock to C₆ to C₁₈ olefins, wherein a higher transitions group metal catalyst is used to metathesize a double bond on a linear portion of the olefin, provided that the double bond is at least three carbons away from a branch if the olefin is branched. The catalyst may include a metal-alkylidene complex and the metal may be ruthenium, osmium, tungsten, or iridium. A suitable catalyst is a Grubbs catalyst. The metathesis process includes a recycle process to maintain the reaction equilibrium, although ethylene can be extracted from the process if a recycle process is not used. A co-solvent is used to increase the product yield. The C₆ to C₁₈ olefin produced by the above process may be suitable for use as drilling fluids.

The invention provides a metathesis process for converting short chain olefins to longer chain olefins.

### BACKGROUND OF THE INVENTION

The market for odd numbered alpha-olefins (C₅, C₇ and C₉) is not yet fully established. As a result, these olefins end up as low value olefins in the fuel pool.

Therefore a need exists to provide a process in which value can be added to these odd numbered olefins. By converting the short chain olefins (C₇ and C₉ olefins) via heterogeneous metathesis (Re, Mo or W-based), longer chain, higher value olefins can be obtained.

Heterogeneous catalysis has been used for the metathesis reaction due to ease of separation of the catalyst and products, ease of regeneration of the catalyst after deactivation, and also for greater thermal stability.

Isomerization of the feed and product, followed by secondary metathesis reactions, is the primary obstacle when dealing with heterogeneous metathesis reactions, as this results in a lower selectivity towards primary metathesis products (Scheme 1). A further problem with the heterogeneous catalysts, however, is the fact that a considerable degree of acidity (both Lewis and Brönsted) is required, which also induces isomerization of the feed and product. The acid sites are required in order to generate the metal-carbene, which is the active species during metathesis. Catalysts with low acidity require an initiator such as Et₃Al or Bu₄Sn to form the metal-carbene, which with consecutive regenerations eventually kills the catalyst due to the formation of a shell around the catalyst.

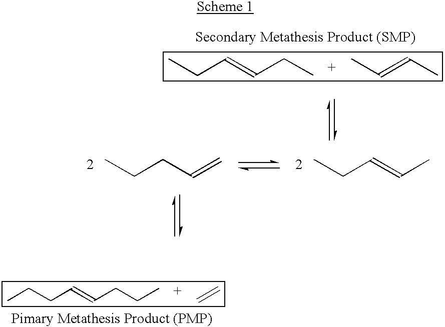

The isomerization that occurs on the catalyst, due to the high degree of acidity, allows for the formation of a great deal of secondary products (Scheme 1). This necessitates the use of a recycle stream in order to convert the unwanted secondary metathesis products to wanted products via metathesis.

It is therefore important that the degree of acidity on the catalyst must be lowered in order to limit the isomerization reactions. Blocking some of the acid sites on the catalyst with alkaline earth metals such as Li⁺, Na⁺, K⁺ or Cs⁺ can achieve this. The result, however, is a significant drop in metathesis activity.

A further disadvantage of classical heterogeneous metathesis (Re-, Mo- or W-based) catalysts is the fact that only olefins without functional groups can be tolerated. Thus extensive feed preparation is required in order to maintain constant metathesis activity.

Therefore a need exists to provide an economically viable metathesis process which is able to efficiently convert short chain olefins to longer chain olefins, without substantial isomerization of the feed and product.

Surprisingly, the inventors have found that a homogeneous metathesis process is capable of converting short chain olefins to longer chain olefins without substantial isomerization of the feed and product occurring during the process. Furthermore, the products formed by the homogeneous metathesis process are formed in superior selectivity towards primary metathesis products compared to the heterogeneous metathesis process.

Homogeneous metathesis was not previously considered because older homogeneous catalysts are extremely air and moisture sensitive, and disposal and downstream treatment is very complicated.

A new generation of homogeneous systems has however been developed which includes structurally well-defined metal-alkylidene complexes which are able to convert highly functionalized and sterically demanding olefins under mild reaction conditions and in high yields. The introduction of these stable alkylidene-metal complexes has significantly expanded the application spectrum of olefin metathesis to organic synthesis.

Homogeneous catalysts have previously been used in polymerization reactions, predominantly ring-opening-metathesis-polymerization (ROMP). ROMP is the most difficult of all metathesis related reactions to accomplish. Any catalyst that is capable of succeeding in this reaction will readily perform normal acyclic metathesis of alpha or internal olefins. The current homogeneous metathesis chemistry is dominated by molybdenum and ruthenium alkylidene complexes.

Mo- and W-based Alkylidene Complexes (Schrock-complexes)

Alkylidene complexes developed by Schrock and Osborn are suitable for application in ROMP of monomers with functional groups.

The stabilized alkylidene-transition metal-complexes are actually initiators as they must first be converted into the actual catalytically active metal-carbene complexes by alkylidene exchange with a double bond. In the case of the catalyst in Scheme 2 the initiation rate is very high. Lewis acids usually associated with homogeneous catalysts and other contaminants are absent in this alkylidene-catalysts making the production of high-purity metathesis products possible. Using the above illustrated alkylidene complexes (Scheme 2) in ROMP, a specific alkene bond can be polymerised, without any side-reactions and minimal polymer decomposition.

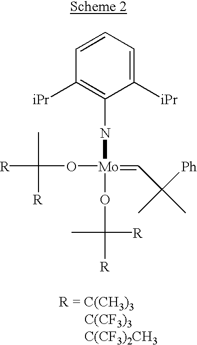

Schrock's catalysts have been studied by several different groups for diverse purposes like the synthesis of highly stereoregular poly-isoprenes via the ROMP of 1-methylcyclobutene. The high selectivity obtained with this catalyst can be attributed to the electrophilicity of the metal center and the steric interaction between the monomer and the metal center. By simply changing the alkoxide groups on the catalyst, it is possible to influence the cis/trans ratio of a product.

Another interesting application of the Schrock complex is in acrylonitrile cross-metathesis. Acrylonitrile is the largest volume organonitrile produced and since organonitrites are versatile synthetic intermediates, acrylonitrile metathesis is a valuable reaction. Conversion of acrylonitrile and a second olefin in the presence of a Schrock Mo-complex within 2-3 h with yields ranging from 40 to 90% is possible, depending on the olefin used for the cross-metathesis reaction.

Together with the Mo-alkylidene complexes, W-catalysts isostructural with the Mo-based catalysts, illustrated in Scheme 2, are also active in the polymerization of compounds like norbornene and boron-containing monomers.

Schrock complexes however have a disadvantage as they have a low tolerance of functional groups and certain reactions have to be performed under strict anhydrous conditions.

Group VIII Metathesis Catalysts

Complexes of ruthenium, osmium and iridium are capable of initiating ROMP. For example, hydrates of RuCl₃, OsCl₃ and IrCl₃ can polymerize norbornene and its derivatives. Anhydrous conditions and exclusion of air are not essential for activity, and indeed, metathesis of 7-oxanorbornene catalyzed by RuCl₃ proceeds in aqueous medium at a higher rate and conversion than in a non-aqueous medium.

Previously used aqueous ruthenium solutions can be used again to initiate additional polymerizations, and furthermore, the catalytic species becomes more active with successive use. Ru(II)complexes like [(C₆H₆)Ru(H₂O)₃]tos₂ (tos=p-toluenesulfonate) behave similarly upon recycling. Thus the key step in the initiation process of Ru(III) is the formation of a Ru(II)olefin complex.

Ru-based Alkylidene Complexes (Grubbs-Complexes)

The knowledge obtained from the investigation of the ruthenium ROMP initiators was applied to the development of the Ru(II) alkylidene complexes (Scheme 3). The ruthenium alkylidene complexes are relatively easy to prepare and handle, tolerate functional groups with O and N atoms, are stable in air and water, are active under mild reaction conditions and display high selectivity.

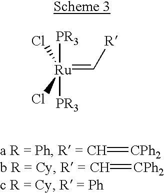

These complexes are also stable in organic solvents, alcohol, acetic acid or a diethyl ether solution of HCl. The use of alkylphosphine ligands makes the catalyst more soluble in organic solvents such as benzene. Water-soluble derivatives can be prepared by phosphine ligand substitution with sterically demanding electron-rich water-soluble phosphines (Scheme 4). This exchange makes the catalyst soluble in both water and methanol.

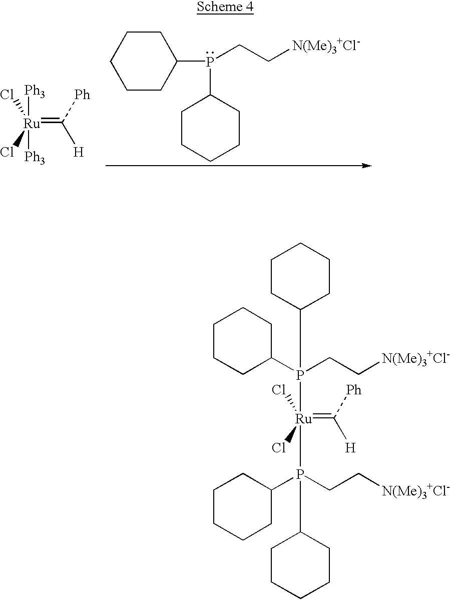

The catalyst illustrated in Scheme 3-b for example is capable of catalyzing the metathesis of functionalized compounds like allyl ether, allyl alcohol and the ring closing metathesis of functionalized dienes.

Substitution of the phosphine ligands on the Grubbs complex in Scheme 3 with N-heterocyclic carbenes (Scheme 5) results in a catalyst that shows high resistance towards functional groups and also reacts faster during ROMP than the previously-known phosphine containing complex. Different N-heterocyclic carbenes can also use subtle steric effects to tune the catalytic performance of the catalyst to obtain more or less of the desired polymer. This steric manipulation is much easier with the N-heterocyclic carbenes than with the known phosphine ligands.

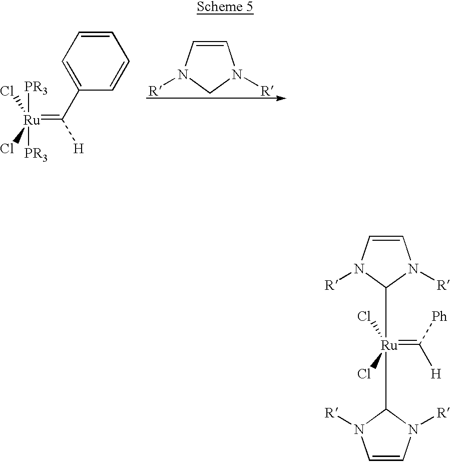

Re-use of Alkylidene Complexes

The alkylidene-metal complexes are, however, expensive and repeated use of the complexes is therefore desirable. For this purpose, the catalyst can be immobilized on a polymeric support. For example, ruthenium-alkylidene complexes bound to polystyrene are significantly more durable than corresponding soluble systems, but unfortunately show lower metathesis rates than the unsupported systems.

Also, most of a Ru-complex (Scheme 6) containing an internal oxygen chelate is recoverable by silica gel column chromatography and can be re-used without any detectable loss in activity.

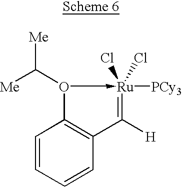

### SUMMARY OF THE INVENTION

According to a first embodiment of the invention there is provided a process for converting C₄ to C₁₀ olefins in a Fischer-Tropsch derived feedstock to C₆ to C₁₈ olefins, the process including a homogeneous metathesis process employing a higher transition group metal catalyst to metathesize a double bond on a linear portion of the olefin, provided that the double bond is at least three carbons away from a branch if the olefin is branched.

The catalyst may include a metal-alkylidene complex and may include tungsten, ruthenium, osmium or iridium catalyst. The catalyst may be a Grubbs catalyst.

The C₄ to C₁₀ olefins may be alpha-olefins, and may be only slightly or not at all isomerized prior to the homogeneous metathesis process.

Pretreatment of the feedstock may be less than pretreatment usually required for heterogeneous metathesis processes.

The olefin products of the homogeneous metathesis process may be formed with increased selectivity compared to the heterogeneous metathesis process.

The feedstock containing the C₄ to C₁₀ olefins may include little or no aromatics or paraffins.

The C₆ to C₁₈ olefins may be linear olefins when the olefin feedstock comprises only linear olefins.

The catalyst may remain active in the presence of impurities in the feedstock, for example oxygenates. More particularly, the catalyst may remain active when oxygenates comprise up to 10% of the feedstock. The catalyst may also be active in the presence of alcohols, aldehydes, ketones and/or acids.

Preferred temperatures for the metathesis process may be from 30 to 150° C., and more preferably the temperature may be 40 to 70° C. The pressure may be maintained from 0 to 30 bar, and more particularly, from 20 to 30 bar.

At least some of the C₆ to C₁₈ olefins produced by the metathesis process may be branched. These olefins may be internal olefins, and more particularly, may be mono-methyl branched internal olefins. The branch may be positioned two or more carbon atoms away from the double bond. Between 0.5% and 10% of the C₆ to C₁₈ olefins may be branched.

The metathesis process may include a recycle process to maintain a reaction equilibrium. Alternatively, ethylene may be extracted from the process to shift the equilibrium in the absence of a recycle process.

A co-solvent may be used during the metathesis process. The co-solvent may be selected so as to increase the product yield of the metathesis process The co-solvent preferably has a polarity scale of between 0.05 and 0.3, and examples of a suitable co-solvent are tetrahydrofurane (THF), diethylether, chlorobenzene, xylene, toluene and alkylated benzene.

The catalyst may be separated from the product-catalyst mixture by short path distillation (SPD), membrane separation, immobilisation on a suitable support carrier, phase separation or solvent extraction.

According to a second embodiment of the invention there is provided a C₆ to C₁₈ olefin, or an isomer, derivative or isotope thereof, produced according to a homogeneous metathesis process substantially as described above.

The C₆ to C₁₈ olefin may be a C₁₄ to C₁₈ olefin formed through the metathesis of at least one of a C₈, C₉ and/or C₁₀ olefin feedstock.

The C₁₄ to C₁₈ olefin may have a double bond positioned in a middle region of the olefin.

The C₆ to C₁₈ olefin may be suitable for use as a drilling fluid.

The olefin feedstock may be derived from a Fischer-Tropsch process or from crude oil.

According to a third embodiment of the invention there is provided a drilling fluid composition derived from olefins having between 14 and 18 carbon atoms, the olefins being obtained by homogeneous metathesis of one or more of a 8, 9 and/or 10 carbon-containing olefin feedstock.

The homogeneous metathesis process may be the process described above.

The olefin feedstock may be derived from a Fischer-Tropsch process.

### DESCRIPTION OF THE INVENTION

The invention will now be described further with reference to the figures and the following non-limiting examples.

In the figures:

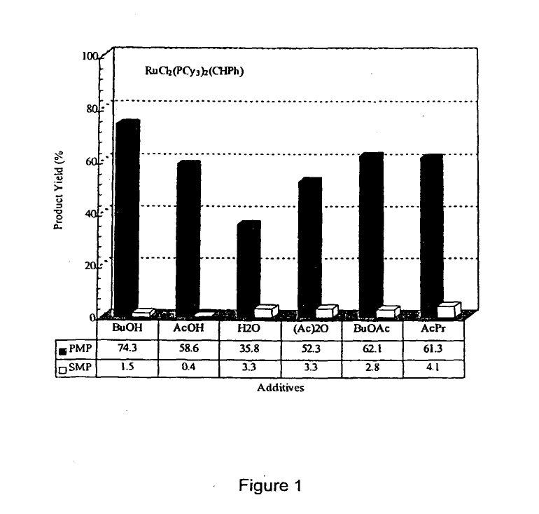

FIG. 1 shows a graph depicting the influence of addatives on the metathesis reaction of 1-octene with a RuCl₂(PCy₃)₂(CHPh) catalyst (Additives/olefin=10%); and

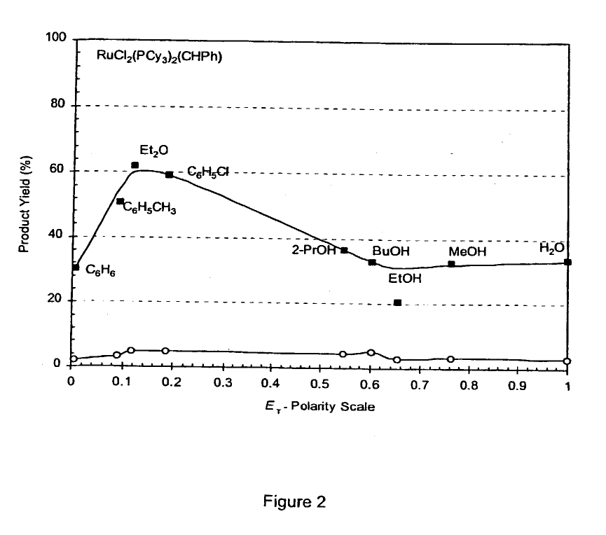

FIG. 2 shows a graph depicting the influence of solvents on the metathesis reaction of 1-octene with a RuCl₂(PCy₃)₂(CHPh) catalyst [(ν)-PMP; (O)-SMP].

A homogeneous catalyst in which the metal-carbene is preformed was used in order to attempt to reduce and preferably to eliminate isomerization of the feed. The “Grubbs” catalyst (RuCl₂(PCy₃)₂CHC₆H₅) was selected as the experimental catalyst due to the fact that this catalyst shows a tolerance towards poisons such as water and other oxygenated compounds.

The Grubbs catalyst was tested on the C₇ stabilized light oil (SLO) narrow cut in order to compare the results with those obtained from two heterogeneous systems (Re and W) previously tested. For this purpose, the Grubbs catalyst was used without any solvent in different ratio's of catalyst to feed at 25° C. (Table 1)

TABLE 1
Grubbs catalyst with C₇ SLO at 25° C. and at equilibrium conversion.

| Catalyst:Feed ratio | Yield C₁₂ |
|---|---|
| 1:100 | 38.5% |
| 1:1000 | 32.2% |
| 1:5000 | 4.1% |
| 1:10000 | 0.4% |

From Table 1, it is apparent that a 1:1000 ratio of catalyst to feed can be used to give a satisfactory Yield of C₁₂. At ratio's above 1:1000, deactivation or inhibition of the catalyst occurs.

The data of the 1:1000 ratio of catalyst to feed (Table 1) can be used as a comparison between the Grubbs catalyst, Re₂O₇/AS40 and WO₃/SiO₂ (Table 2).

TABLE 2
Comparison between the Grubbs catalyst, Re-based catalyst and
W-based catalyst, using C₇ SLO as feed

| Catalyst | Temp. | Yield PMP | Select. PMP | C₁₂ linearity |
|---|---|---|---|---|
| Re₂O₇/AS40 | 80° C. | 14.5% | 20.7% | 81.9% |
| WO₃/SiO₂ | 500° C. | 7.8% | 8.6% | 76.4% |
| Grubbs catalyst | 25° C. | 37.6% | 98.2% | 91.0% |

PMP = Primary metathesis products (C₁₂ + C₂)
*Recycle of C₅-C₈ in order to optimize yield towards C₉+

The low selectivity towards the primary metathesis products in the heterogeneous systems (Re and W) can be explained in terms of the high degree of isomerization of the feed, followed by metathesis to yield secondary metathesis products.

It is thus apparent that homogeneous systems can be advantageous over the heterogeneous systems with respect to PMP.

Homogeneous metathesis of C₇ SLO resulted in a narrow product range, containing almost exclusively 6-dodecene and mono-methyl branched 5-undecenes (as per GC-MS analysis). This results in much higher yields towards the C₁₂ fraction (Table 2) as compared to the metathesis reactions in which heterogeneous catalysts were employed.

In order to determine the effect of “poisons” on the metathesis reaction, 1-octene (99+ % pure) was used and the feed was spiked with various contaminants. A summary of the results is shown in FIG. 1.

The RuCl₂(PCy₃)₂(CHPh) catalyst showed almost no deactivation in the presence of additives. All of the reactions reached equilibrium with 10% additives added. It was only with the addition of H₂O that little deactivation was detected after 2 h. BuOH showed an increase in activity and a yield of 74% primary metathesis and about 1% of secondary metathesis products were obtained.

A study on the influence of solvents during the homogeneous metathesis reaction was conducted (FIG. 2). From FIG. 2, it is apparent that the polarity of the solvent affects the product yield, which can almost be tripled if a suitable solvent is selected.

Formation of a C₁₆-C₁₈ olefin range, where the double bond is exactly in the middle of the molecule, was possible due to the much narrower range of products produced by the homogeneous metathesis reactions as compared to the range of products produced by heterogeneous metathesis reactions. The C₁₆-C₁₈ olefin cut was formed through the metathesis of a C₉ and C₁₀ alpha olefin mixture. This cut is very low in aromatic and diene content, which makes it suitable for application as a drilling fluid.

The invention is not limited to the precise constructional details as herein described.

The applicant believes that the invention is advantageous in that it provides a process for transforming C₄ to C₁₀ olefins into a narrow range of higher value longer chain products. The products are furthermore formed with increased selectivity than in heterogeneous metathesis processes.

1. A homogeneous metathesis process for converting C₄ to C₁₀ olefins in a Fischer-Tropsch derived feedstock to C₆ to C₁₈ olefins, wherein a higher transition group metal catalyst is used to metathesize a double bond on a linear portion of the olefin, provided that the double bond is at least three carbons away from a branch if the olefin is branched.

2. A metathesis process as claimed in claim 1, wherein the catalyst includes tungsten, ruthenium, osmium or iridium.

3. A metathesis process as claimed in either of claims 1 or 2, wherein the catalyst includes a metal-alkylidene complex.

4. A metathesis process as claimed in any one of claims 1 to 3, wherein the catalyst is a Grubbs catalyst.

5. A metathesis process as claimed in any one of claims 1 to 4, wherein the C₄ to C₁₀ olefins are alpha-olefins.

6. A metathesis process as claimed in any one of claims 1 to 5, wherein the C₄ to C₁₀ olefins are only slightly or not at all isomerized prior to the homogeneous metathesis process.

7. A metathesis process as claimed in any one of claims 1 to 6, wherein the feedstock containing the C₄ to C₁₀ olefins includes little or no aromatics or paraffins.

8. A metathesis process as claimed in any one of claims 1 to 7, wherein at least some of the C₆ to C₁₈ olefins produced by the metathesis process are branched.

9. A metathesis process as claimed in claim 8, wherein the C₆ to C₁₈ olefins are internal olefins.

10. A metathesis process as claimed in claim 9, wherein the C₆ to C₁₈ olefins are mono-methyl branched internal olefins.

11. A metathesis process as claimed in any one of claims 8 to 10, wherein the branch is positioned two or more carbon atoms away from the double bond.

12. A metathesis process as claimed in any one of claims 8 to 11, wherein between 0.5% and 10% of the C₆ to C₁₈ olefins are branched.

13. A metathesis process as claimed in any one of claims 1 to 7, wherein the C₆ to C₁₈ olefins are linear olefins when the olefin feedstock comprises only linear olefins.

14. A metathesis process as claimed in any one of claims 1 to 13, wherein the feedstock includes oxygenates, alcohols, aldehydes, ketones and/or acids.

15. A metathesis process as claimed in any one of claims 1 to 14, wherein up to 10% of the feedstock is comprised of oxygenates.

16. A metathesis process as claimed in any one of claims 1 to 15, wherein the reaction temperature is from 30 to 150° C., and the pressure is from 0 to 30 bar.

17. A metathesis process as claimed in claim 16, wherein the temperature is from 40 to 70° C.

18. A metathesis process as claimed in either of claims 16 or 17, wherein the pressure is from 20 to 30 bar.

19. A metathesis process as claimed in any one of claims 1 to 18, which includes a recycle process to maintain a reaction equilibrium.

20. A metathesis process as claimed in claim 18, wherein ethylene is extracted from the process to shift the equilibrium in the absence of a recycle process.

21. A metathesis process as claimed in any one of claims 1 to 20, wherein a co-solvent is used to increase the product yield.

22. A metathesis process as claimed in claim 21, wherein the co-solvent has a polarity scale of between 0.05 and 0.3.

23. A metathesis process as claimed in either of claims 21 or 22, wherein the co-solvent is tetrahydrofurane (THF), diethylether, chlorobenzene, xylene, toluene or alkylated benzene.

24. A metathesis process as claimed in any one of claims 1 to 23, wherein the catalyst is separated from the product-catalyst mixture by short path distillation (SPD), membrane separation, immobilisation on a suitable support carrier, phase separation or solvent extraction.

25. A C₆ to C₁₈ olefin, or an isomer, derivative or isotope thereof, produced according to a homogeneous metathesis process as claimed in any one of claims 1 to 24.

26. A C₆ to C₁₈ olefin as claimed in claim 25, wherein the olefin is a C₁₄ to C₁₈ olefin formed through the metathesis of at least one of a C₈, C₉ and/or C₁₀ olefin feedstock.

27. A C₆ to C₁₈ olefin as claimed in claim 26, wherein the C₁₄ to C₁₈ olefin has a double bond positioned in a middle region of the olefin.

28. A C₆ to C₁₈ olefin as claimed in any one of claims 25 to 27, which is suitable for use as a drilling fluid.

29. A C₆ to C₁₈ olefin as claimed in any one of claims 25 to 28, wherein the olefin feedstock is derived from a Fischer-Tropsch process or from crude oil.

30. A drilling fluid composition derived from olefins having between 14 and 18 carbon atoms, the olefins being obtained by homogeneous metathesis of one or more of a 8, 9 and/or 10 carbon-containing olefin feedstock.

31. A drilling fluid composition as claimed in claim 30, wherein the homogeneous metathesis process is the process described in claims 1 to 24.

32. A drilling fluid composition as claimed in either of claims 30 or 31, wherein the olefin feedstock is derived from a Fischer-Tropsch process.

33. A homogeneous metathesis process according to the invention for converting C₄ to C₁₀ olefins in a feedstock to C₆ to C₁₈ olefins, substantially as hereinbefore described and exemplified.

34. A homogeneous metathesis process for converting C₄ to C₁₀ olefins in a feedstock to C₆ to C₁₈ olefins including any new and inventive integer or combination of integers, substantially as herein described.

35. A C₆ to C₁₈ olefin as claimed in any one of claims 25 to 29, substantially as hereinbefore described and exemplified.

36. A C₆ to C₁₈ olefin including any new and inventive integer or combination of integers, substantially as herein described.

37. A drilling fluid composition as claimed in any one of claims 30 to 32, substantially as hereinbefore described and exemplified.

38. A drilling fluid composition including any new and inventive integer or combination of integers, substantially as herein described.
